(19) United States
(12) Patent Application Publication
(10) Pub. No.: US 2026/0013715 A1
(43) Pub. Date: Jan. 15, 2026

(54) AIR SPECULUM

(71) Applicant: Ivy Next Ltd., Caesarea Industrial Park (IL)

(72) Inventors: Dean Cohen, Carmiel (IL); Kfir Solomon, Zoran (IL); Sefi Shachrur, Pardes-Hana Karkur (IL); Gil Bachar, Tel Aviv (IL)

(73) Assignee: Ivy Next Ltd., Caesarea Industrial Park (IL)

(21) Appl. No.: 18/772,535

(22) Filed: Jul. 15, 2024

(51) Int. Cl. A61B 1/32; A61B 1/303

(52) U.S. Cl. CPC A61B 1/32; A61B 1/303

(57) ABSTRACT

A speculum includes a handle including a pressurized gas flow lumen, and a vaginal interface element coupled to a distal end of the housing, which has a flexible cup with folds and an air inlet septum. A probe having an imaging lumen passes through the vaginal interface element. The imaging lumen includes an optical window, a viewing device positioned proximal to the optical window, and a biasing device configured to urge the viewing device to abut against an inner surface of the optical window.

### FIELD OF THE INVENTION

The present invention relates to vaginal speculums and particularly to a vaginal speculum that uses pressurized gas to maintain a passageway through the vagina, such as to gain access to the cervix or other anatomy.

### BACKGROUND OF THE INVENTION

PCT Patent Application PCT/IB2020/053647 (WO 2020/212923) describes an air speculum. The speculum includes a speculum sheath formed with a first lumen which is in fluid communication with a pump and controller unit for introducing pressurized air into a vagina. A second lumen is in fluid communication with the pump and controller unit for measuring air pressure in the vagina. A third lumen is provided for a viewing device.

PCT Patent Application PCT/IB2021/060539 (WO 2022/101864) describes another air speculum, which includes a vaginal interface element which is a soft, flexible cup with folds. The vaginal interface element may be pressed against the labia minora and/or labia majora of the vagina so as to reduce and flow of material outwards.

PCT Patent Application PCT/IB2023/062489 describes another air speculum, which includes a speculum sheath formed with one or more fluid flow lumens in fluid communication with a pump and controller unit for introducing pressurized fluid into a vagina, a pressure sensor lumen in fluid communication with the pump and controller unit for measuring fluid pressure in the vagina, and a third lumen in which is disposed a viewing device. A vaginal interface element includes flexible folds and is configured for pressing and sealing against a vagina. The vaginal interface element includes one or more pressure relief exit ports.

### SUMMARY OF THE INVENTION

The present invention seeks to provide an air speculum with added features to those of the air speculum described in PCT Patent Application PCT/IB2023/062489, as described in detail below.

There is thus provided in accordance with a non-limiting embodiment of the present invention a speculum including a handle including a pressurized gas flow lumen, a vaginal interface element coupled to a distal end of the housing, the vaginal interface element including a flexible cup with folds and an air inlet septum, and a probe having an imaging lumen that passes through the vaginal interface element, wherein the imaging lumen includes an optical window, a viewing device positioned proximal to the optical window, and a biasing device configured to urge the viewing device to abut against an inner surface of the optical window. The probe may also include a tool channel.

In accordance with a non-limiting embodiment of the invention an opaque ring is placed at a distal face of the imaging lumen, the opaque ring surrounding the optical window.

In accordance with a non-limiting embodiment of the invention the optical window is spaced proximally from a distal face of the imaging lumen.

In accordance with a non-limiting embodiment of the invention an air opening is located around the probe, and wherein a pressurized gas is flowable from the pressurized gas flow lumen to the vaginal interface element and then flowable through the air opening into a vaginal canal.

In accordance with a non-limiting embodiment of the invention viewing device is operative to capture images at different focal planes in a vagina or a cervix.

### BRIEF DESCRIPTION OF THE DRAWINGS

The present invention will be understood and appreciated more fully from the following detailed description, taken in conjunction with the drawing in which:

FIGS. 1 and 2 are simplified pictorial and sectional illustrations, respectively, of an air speculum, in accordance with a non-limiting embodiment of the present invention, with a flexible vaginal interface element.

FIG. 3 is a simplified sectional illustration of the air speculum, showing an imaging lumen, a tool channel, and an optical window.

FIG. 4 is a simplified end-view illustration of the air speculum, showing the imaging lumen, the tool channel, and the flexible vaginal interface element.

FIG. 5 is an enlarged illustration of the imaging lumen, showing an opaque ring at the distal face of the imaging lumen and the clear, transparent optical window.

FIG. 6 is a simplified illustration of the different focal planes in the vagina or cervix.

FIG. 7 is an illustration of a display of the different focal planes imaged by the system.

### DETAILED DESCRIPTION OF EMBODIMENTS

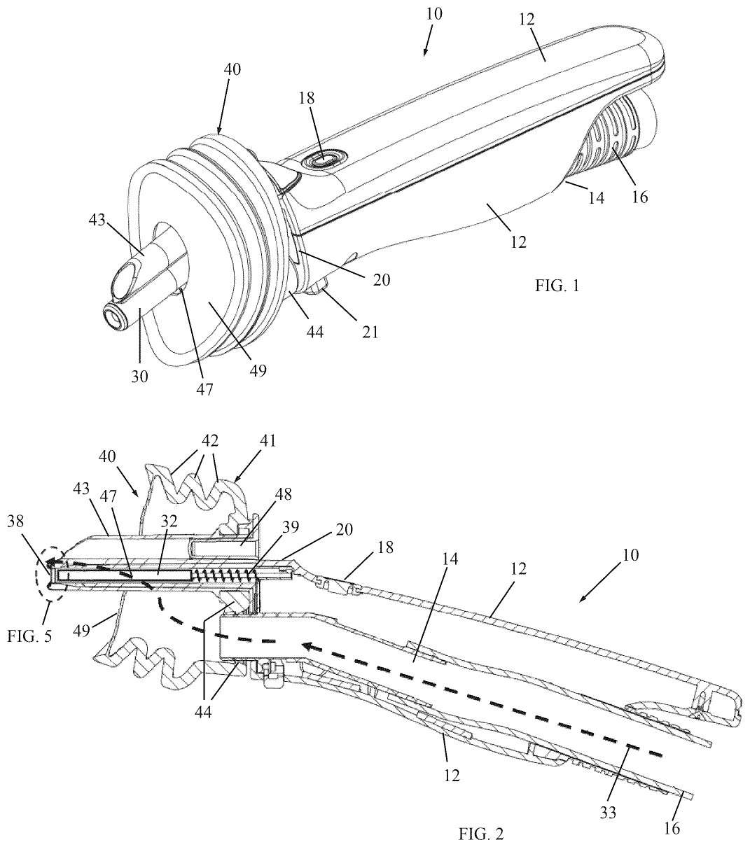

Reference is now made to FIGS. 1 and 2, which illustrate an air speculum frontend 10, constructed and operative in accordance with a non-limiting embodiment of the present invention. FIG. 2 shows a section of the air speculum front end shown in FIG. 1.

Speculum 10 includes a handle 12, which may include a pressurized gas flow lumen 14, into which a pressurized gas tube 16 may be inserted. Pressurized gas tube 16 is fluidly connected to a pressurized gas source (not shown). Handle 12 may include one or more controls 18, such as an on/off, pressure increase/decrease, flow increase/decrease, buttons and other controls over flow supply or any other parameters of the system described below. The handle 12 of the air speculum 10 may be coupled to a backend server (not shown) that provides air flow 33 (through gas tube 16 and into flow lumen 14) and electric power, and which has a controller that receives camera images and other digital inputs (e.g., flow meter readings, control commands, etc.) and regulates air flow 33.

Handle 12, may further include a viewing device 32. The viewing device 32 may include, without limitation, a small diameter tube camera (e.g., 4-6 mm external diameter) with a wide lens (90°-120° or 90-180°), good resolution (e.g. 2-8 MPxl or spatial resolution of at least 1 μm or at least 10 μm on the subject) and means to transfer imaged data to a controller (e.g. MIPI interface, coax connection, USB connection or other connections known in the art). The small diameter tube camera may be a fixed focus camera or an autofocus camera comprising a means (e.g. VCM (voice coil motor) actuator) to shift the lens of the camera relative to the camera sensor.

A distal end 20 of handle 12 is configured to be coupled to a vaginal interface element 40, described below. Vaginal interface element 40 may be connected and/or detached (released) from handle 12. Handle 12 may include a button 21 (FIG. 1) to securely connect and/or release vaginal interface element 40. Handle 12 is preferably reusable, whereas vaginal interface element 40 is preferably disposable, but alternatively both could be disposable or reusable, in which case vaginal interface element 40 would be sterilized, disinfected or cleaned before each use, according to known medical standards.

The vaginal interface element 40 may include a soft, flexible cup 41 with folds 42, shaped as a bellows, for example. Folds 42 are designed to withhold the pressure of the air speculum and/or handle 12 against the vagina of a female patient. In a preferred embodiment, without limitation, folds 42 are designed to contract under compression by 5-15 mm due to a pressure of 5-20 newton force. The vaginal interface element 40 may include an air inlet septum 44, through which the distal end of pressurized gas tube 16 may be inserted, so that pressurized gas can enter vaginal interface element 40, as designated by reference numeral 33 in FIG. 2. The air inlet septum 44 seals vaginal interface element 40 against flow lumen 14. The air inlet septum 44 may be offset from the longitudinal central axis of vaginal interface element 40. Folds 42 may be asymmetric with respect to the longitudinal central axis of vaginal interface element 40; as seen, the folds 42 may be more compressed on one side of the longitudinal central axis as opposed to the opposite side. The folds 42 may be slanted (not perpendicular) with respect to the longitudinal central axis of vaginal interface element 40. Folds 42 allow the application of pressure of the handle and cup on the patient's body to seal against air leaks.

An imaging lumen 30 may pass through vaginal interface element 40. Imaging lumen 30 may be rigid and designed to enter the vaginal canal of a female. The imaging lumen may extend distally beyond the flexible cup of vaginal interface. In a preferred embodiment, imaging lumen 30 extends distally beyond the flexible cup of vaginal interface, without limitation, by 2-5 cm, or by 3-4.5 cm. The imaging lumen 30 may house or accommodate the viewing device. In one example, imaging lumen 30 may have an internal diameter larger than the small diameter camera, e.g., without limitation, an internal diameter larger than 6 mm or larger than 7 mm and smaller than 10 mm. Imaging lumen 30 external diameter may be smaller than 12 mm or smaller than 11.5 mm or smaller than 11 mm, such that it can enter easily into a female vagina.

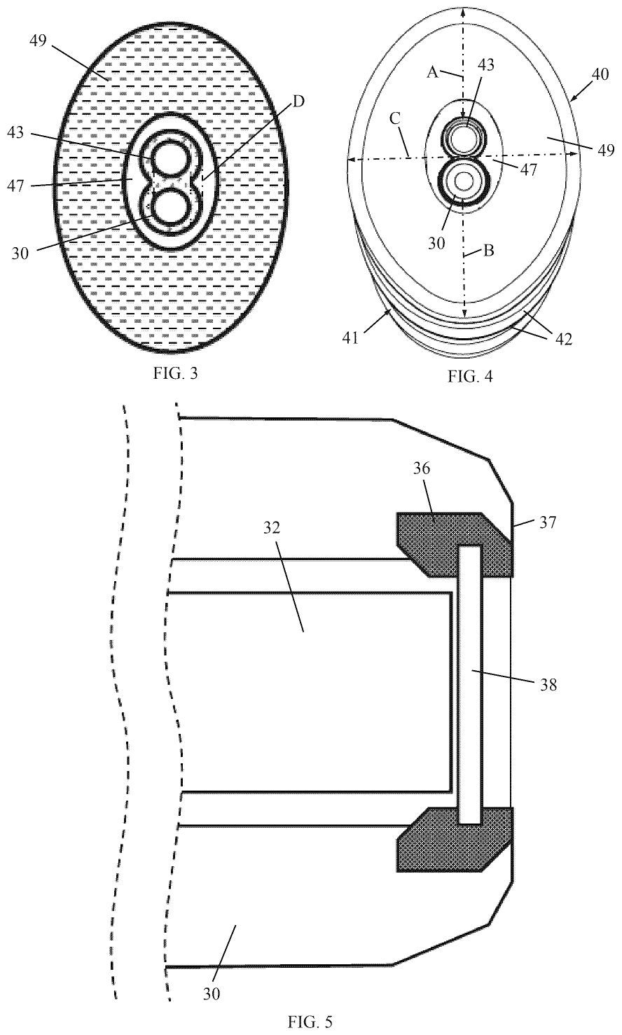

Reference is now made to FIG. 5. The imaging lumen 30 may be a transparent light guide made of a transparent material, such as but not limited to, polycarbonate, polyethylene, PMMA (poly(methylmethacrylate)), or other engineering plastics. An opaque ring 36 (e.g., a black ring) may be placed at the distal face 37 of imaging lumen 30 to reduce glare effects from the internal illumination. The opaque ring 36 may surround a clear, transparent optical window 38. Optical window 38 may be made from a transparent plastic such as but not limited to, polycarbonate, polyethylene, PMMA or other engineering plastics or from glass. The optical window 38 is preferably proximal to the distal face 37, such as, without limitation, by a distance of 1.2-6 mm, which reduces or completely prevents the window 38 from contacting mucus, tissues, fluids or other debris inside the patient's vagina. In this application, “distal” means toward the patient side and “proximal” means toward the air input side.

Referring again to FIG. 2, it is seen that viewing device 32 may be biased by a biasing device 39, such as a coil spring, which urges viewing device 32 to abut against the inner surface of optical window 38. The biasing device 39 reduces the distance between viewing device 32 and window 38 to zero (thus compensating for any distance caused by manufacturing variance in plastic moldings) and thereby increases the optical field of view of viewing device 32. In an embodiment, pressing button 21 (described above) will release vaginal interface element 40, while biasing device 39 presses vaginal interface element 40 away from handle 12, allow disposing vaginal interface element 40 without having to touch it by hand, which may help reduce post examination contaminations.

As seen in FIG. 2, a tool channel (lumen) 43 may pass through vaginal interface element 40. Tool channel 43 may include a tool channel plug 48 that seals the passage of a tool (such as but not limited to, cutting tools, drug delivery tools, grasping tools, Pap smear test stick, etc.) through vaginal interface element 40. Plug 48 may be removed and tools may be introduced into the working channel.

The vaginal interface element 40 may include one or more pressure relief exit ports (not shown). When vaginal interface element 40 is pressed against the labia minora and/or labia majora and/or vulva of the vagina and pressurized gas is introduced to inflate the vaginal canal, the pressure in the inflated vagina up to the cervix is about equal to the pressure in the inner volume of vaginal interface element 40. This pressure is greater than the external atmospheric pressure. When vaginal interface element 40 is moved away from the labia minora and/or labia majora of the vagina and/or vulva, unwanted air flow sounds may be caused. The pressure relief exit ports prevent such unwanted sounds by venting some of the pressurized gas.

FIGS. 3 and 4 illustrate the vaginal interface element 40 in a front view. FIG. 3 is a sectional view and FIG. 4 is an end view. In an embodiment, without limitation, the distance of the top part of flexible cup 41 from probe is 20-45 mm (marked A in FIG. 4). In an embodiment, without limitation, the distance of the bottom part of flexible cup 41 from the probe is 20-30 mm (marked B in FIG. 4). A may be equal to B or they may differ in size. In an embodiment, without limitation, the front face width of flexible cup 41 is 45-55 mm or 40-60 mm wide (marked C in FIG. 4). In the front view the tool channel 43 and imaging lumen 30 are seen. A membrane 49, which may be made of a soft polymer or other suitable material, covers the distal end of the vaginal interface element 40, slightly inwards of the end of the flexible cup. The imaging lumen 30 and tool channel 43 pass through an opening in membrane 49. This opening, called air opening 47, allows air to flow past the sides of imaging lumen 30 and tool channel 43. The two channels are designed tangent to one another. Air opening 47 is preferably, although not necessarily, narrow to follow the outer contours of imaging lumen 30 and tool channel 43; thus, since they are tangent to each other, air opening 47 may take on the shape of the number “8”. Other shapes are also within the scope of the invention. In general, there is a probe, which includes imaging lumen 30 and which may additionally include tool channel 43) that passes through the opening in membrane 49. The probe has a cross section in the plane perpendicular to the optical axes of the camera that is concave (in the case of a figure “8” shape, the concave shapes are at the middle of the 8-shape where the upper and lower circles of the “8” meet each other). This concave shape allows air to flow from the cup into the vagina. In an embodiment, without limitation, the area trapped in the concave part of the cross section of the probe (or the sum of areas in case of a few concave parts as seen in FIG. 3 and marked with D) is 5-15 square mm.

As seen in FIGS. 2 and 4, the pressurized gas 33 flows through air opening 47 located around the outer contours of imaging lumen 30 and tool channel 43. Accordingly, pressure is increased through the air speculum 10 in two stages. First, the pressurized gas 33 flows from the pressurized gas source to and through the pressurized gas tube 16 located in the pressurized gas flow lumen 14 and flows to fill the inner volume of vaginal interface element 40. Next, the pressurized gas 33 flows through air opening 47 into the vaginal canal to build up pressure in the vaginal canal.

Inflation gas may be used to inflate the vaginal canal at least to 5 mmHg and up to 20 mmHg or up to 30 mmHg, without limitation. The inflation gas may be, without limitation, air, CO₂ or nitrogen, for example. In an embodiment a gas pressure sensor (not seen) may be located in or on handle 12 to measure the real-time pressure in the inner volume of vaginal interface element 40. A controller (not seen) receives the reading of pressure sensor and controls the flow rate of pressurized gas 33. A pressure relief valve (not seen) may be employed for safety. The pressure of the inner volume of vaginal interface element 40 may be sensed by the pressure sensor and sent to a control unit that operates in a control loop feedback for optimal safety and efficiency.

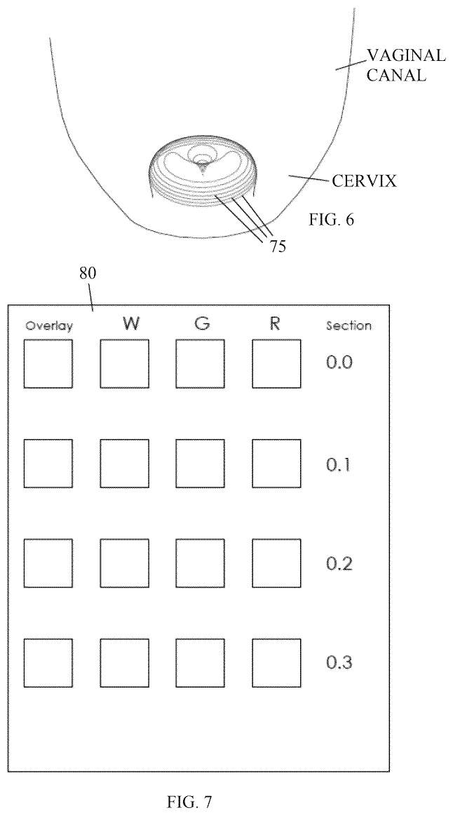

FIG. 6 illustrates the different focal planes 75 in the vagina or cervix, which are imaged by the viewing device.

According to one non-limiting example of operation, the system for vaginal imaging may operate as follows:
a. Focus on the cervix or vagina, for example on the most proximal plane of focus or the furthest plane of focus.
b. Change the focus plane to image the cervix or vagina in several focal planes, for example every 50 or 100 micrometers, or for example, on 5-15 focal planes from the most proximal to the furthest focal plane.
c. Provide the user an image of the cervix or the vagina in all focal planes.

According to another non-limiting example, the vagina or cervix may be imaged in multispectral imaging. In one case, the illumination in the vaginal canal may be changed in different wavelengths: blue, green, red, deep-red, infra-red, etc., and the images may be combined to a single multicolor image or shown to the user in several panels.

FIG. 7 illustrates a non-limiting exemplary display 80 of the different focal planes and different color schemes imaged by the system. Such a display may aid the user to identify medical abnormalities (e.g. lesions, scars, fungi etc.).

What is claimed is:

1. A speculum comprising:
a handle comprising a pressurized gas flow lumen and a viewing device;
a vaginal interface element coupled to a distal end of said housing, said vaginal interface element comprising a flexible cup with folds and an air inlet septum; and
a probe which comprises an imaging lumen and which passes through said vaginal interface element, wherein said imaging lumen comprises an optical window, wherein said viewing device is positioned proximal to said optical window, and a biasing device is configured to urge said viewing device to abut against an inner surface of said optical window.

2. The speculum according to claim 1, wherein the probe further comprises a tool channel.

3. The speculum according to claim 1, wherein an opaque ring is placed at a distal face of said imaging lumen, said opaque ring surrounding said optical window.

4. The speculum according to claim 1, wherein said optical window is spaced proximally from a distal face of said imaging lumen.

5. The speculum according to claim 4, wherein said optical window is spaced proximally from a distal face of said imaging lumen by a distance of 1.2-6 mm.

6. The speculum according to claim 1, wherein an air opening is located around said probe, and wherein a pressurized gas is flowable from said pressurized gas flow lumen to said vaginal interface element and then flowable through said air opening into a vaginal canal.

7. The speculum according to claim 1, wherein said imaging lumen extends distally beyond the flexible cup said vaginal interface by 3-4.5 cm.

8. The speculum according to claim 1, wherein said imaging lumen extends distally beyond the flexible cup said vaginal interface by 2-5 cm.

9. The speculum according to claim 1, wherein said probe has a concave shaped cross section.

10. The speculum according to claim 1, wherein said probe has an “8” shaped cross section.

11. The speculum according to claim 1, wherein said flexible cup is designed to contract under compression by 5-15 mm due to a pressure of 10 newton force.

12. The speculum according to claim 1, wherein said vaginal interface is detachable from said handle.

13. The speculum according to claim 1, wherein said viewing device is operative to capture images at different focal planes in a vagina or a cervix.

14. The speculum according to claim 1, wherein the vaginal interface further comprises a vaginal facing membrane, designed to seal against a vulva of a female.

15. The speculum according to claim 1, wherein the vaginal interface imaging lumen is designed to enter a vaginal canal of a female.

16. A method for vaginal imaging comparing the following steps:
a. focusing on a focus plane at or near a cervix or a vagina;
b. changing the focus plane to image the cervix or the vagina in several focal planes; and
c. providing images of the cervix or the vagina in all focal planes.

17. The method according to claim 16, wherein the focus plane is the most proximal plane of focus or the furthest plane of focus in the cervix or the vagina.

18. The method according to claim 16, wherein the several focal planes comprise planes located every 50 or 100 micrometers from the most proximal to the furthest focal plane in the cervix or the vagina.

19. The method according to claim 16, wherein the several focal planes comprise 5-15 focal planes from the most proximal to the furthest focal plane in the cervix or the vagina.
